(19) United States
(12) Patent Application Publication
(10) Pub. No.: US 2024/0400973 A1
(43) Pub. Date: Dec. 5, 2024

(54) METHOD AND SYSTEM FOR PRODUCING EUGLENA BIOMASS WITH CONTROLLED BIOMASS COMPOSITIONS

(71) Applicant: Geb Impact Technology Company Limited, Hong Kong (HK)

(72) Inventors: Ting Hin Kosmo YAN, Hong Kong (HK); Shui Ying HIE, Hong Kong (HK); Zhonghui Alex WANG, Hong Kong (HK)

(21) Appl. No.: 18/326,446

(22) Filed: May 31, 2023

(51) Int. Cl. C12N 1/12; C12M 1/00

(52) U.S. Cl. CPC C12N 1/12; C12M 29/18; C12M 29/26

(57) ABSTRACT

Disclosed are a method and system for producing axenic Euglena biomass with controlled biomass compositions, the method including the steps of: modifying components in an aqueous culture medium; culturing the Euglena using the modified aqueous medium under at least one culture condition in a fed-batch culture system; and harvesting the biomass when a desired amount of the biomass compositions of interest is achieved.

### FIELD OF THE INVENTION

The present invention relates to a method of culturing Euglena under various conditions to produce Euglena biomass compositions with high productivity.

### BACKGROUND OF THE INVENTION

Aquatic single-celled plant microorganisms, known as microalgae, live individually or in groups in freshwater or saltwater. They grow much faster than land plants, especially when provided with sufficient nutrients, light, and carbon dioxide. Therefore, microalgae are well-suited for absorbing atmospheric CO₂, producing biofuel, purifying wastes, and generating biomolecules for various purposes.

Various species of microalgae are currently cultivated industrially for producing food supplements, pigments, feeds, omega 3 fatty acids, biomass for aquaculture, and wastewater treatment. The cultivation process takes place in basins, tanks, photobioreactors, and fermenters, with different techniques and volumes depending on the species and intended applications. Although microalgae cultivation for industrial purposes has several promising features, certain critical factors still impede their full potential utilization despite considerable research and development efforts.

An example of a commonly available microalgae is Euglena gracilis (E. gracilis) which is a unicellular freshwater microalga that belongs to the genus Euglena. It is a versatile organism that can be easily grown and is known for its ability to produce various compounds such as paramylon, lipids, and proteins. Paramylon is a unique carbohydrate found in Euglena gracilis that functions as a storage compound. It is a highly branched beta-1,3-glucan that can account for up to 70% of the alga's dry weight. Paramylon has a complex structure and is known to have various biological activities such as antioxidant, immunostimulatory, and antitumor effects. Euglena gracilis is also known for its ability to accumulate lipids, especially under stress conditions. The lipid content can account for up to 30% of the alga's dry weight. The fatty acid composition of the lipids varies depending on the growth conditions, but it is generally rich in polyunsaturated fatty acids (PUFAs) such as eicosapentaenoic acid (EPA) and docosahexaenoic acid (DHA). These PUFAs have various health benefits and are used in the production of nutraceuticals, functional foods, and pharmaceuticals.

In addition to paramylon and lipids, Euglena gracilis is also a rich source of proteins. The protein content of Euglena gracilis can range from 40% to 70% of the alga's dry weight, depending on the growth conditions. The protein is of high quality, with a well-balanced amino acid composition and a high digestibility. Euglena gracilis proteins have potential applications in various fields such as food, feed, and biotechnology.

E. gracilis had reportedly been cultivated under different trophic modes, including photoautotrophic (PT) under illumination of light, heterotrophic (HT) in the dark environment and mixotrophic mode (MT) specifically known as photoheterotrophic cultivation. Among these reports, the biomass productivities were reported to be ranging from <1 g/L under autotrophic mode, to ˜86 g/L by mixotrophic-heterotrophic mode with low protein content (16% dry basis). Another study has shown that by tuning the pH of stationary phase culture, the protein content comprised 45% of dry biomass (DBM), however the productivity is still relatively low (8 g/L). These different studies independently show that the protein, paramylon, and lipids in biomass are highly variable with different cultivation techniques, suggesting the high plasticity of nutritional composition of Euglena.

United States Patent No. 20200231923 A1 disclosed a method for the simultaneous heterotrophic and mixotrophic cultivation of microalgae, to a system for the simultaneous heterotrophic and mixotrophic cultivation of microalgae and to the use of the method and/or the system for the cultivation of microalgae. It is noted that the above-mentioned system allows the cultivation of microalgae under heterotrophic and mixotrophic conditions. Whilst it results in high biomass densities and high time yields, the said method and system may not be able to produce Euglena biomass in high productivity under shorter cultivation period. The system may not be able to monitor, retain, and ensure high productivity of biomass efficiently with controlled biomass compositions such as paramylon, protein, and lipid.

Japan Patent Application No. 6113819A disclosed a method for culturing Euglena, and more particularly to a method for culturing Euglena having a high protein content. It is stated that paramylon is accumulated in the cells of Euglena cells during the late logarithmic growth phase or stationary phase, and further converted into protein wherein the conversion efficiency depends on the specific medium pH. The conversion of paramylon to protein was efficiently carried out by keeping the nitrogen source in the medium from being depleted in the late logarithmic growth phase to the stationary phase. Also, the Euglena cells can be cultured under light irradiation or dark culture to obtain high protein contents. Whilst the above method is able to obtain high protein content, the said method will not be able to predict the amount of protein, paramylon, and lipid. At the same time, the method may not be able to monitor, retain and ensure high productivity of biomass efficiently.

U.S. Pat. No. 9,758,756 B2 disclosed a method of culturing microorganisms in combinations of phototrophic, mixotrophic, and heterotrophic culture conditions. A culture of microorganisms may be transitioned between culture conditions over the life of a culture in various combinations, utilizing various conditions in a sequential manner to optimize the culture for growth or product accumulation. The technology utilized in this patent will not be able to predict the biomass compositions such as the carbohydrates, lipids and protein contents.

Therefore, there is a need to have a method and system that enable high productivity of Euglena biomass, at the same time maintaining carbohydrate, preferably the paramylon, protein, and lipid contents efficiently. Additionally, the said method must allow flexibility in order to customize and tune up the production of the protein, paramylon and/or lipid whenever required and the said method must be cost-effective and time saving.

### BRIEF SUMMARY OF THE INVENTION

It is an objective of the present invention to produce Euglena biomass with controlled biomass compositions under controlled environment with high productivity and efficiency in a short amount of time.

It is also an objective of the present invention to provide a method with flexibilities and ease of modifications in order to produce Euglena biomass with controlled biomass compositions whilst maintaining high productivity and efficiency.

Accordingly, these objectives can be achieved by following the teachings of the present invention, which relates to a method and system for producing axenicEuglena biomass with controlled biomass compositions, the method comprising the steps of: modifying components in an aqueous culture medium; culturing the Euglena using the modified aqueous medium under at least one culture condition in a fed-batch culture system; and harvesting the biomass when a desired amount of the controlled biomass compositions of interest is achieved.

### BRIEF DESCRIPTION OF THE SEVERAL VIEWS OF THE DRAWINGS

The features of the invention will be more readily understood and appreciated from the following detailed description when read in conjunction with the accompanying drawings of the preferred embodiment of the present invention, in which:

FIG. 1 illustrates the flow chart of the sequence of cultivation process to produce different Euglenabiomass compositions with preferred culture conditions;

FIG. 2 illustrates the relationship of paramylon and protein content in Euglena dry biomass harvested from different cultivation conditions of different medium tuning and cultivation period;

FIG. 3 illustrates the time course analysis of paramylon content and biomass productivity of harvested biomass from day 11 to day 17 of prolonged cultivation from FIG. 2;

FIG. 4 illustrates paramylon contents and biomass productivities of Euglena cultures that were fed with total nutrient (4×CM+4% glucose) and continuously illuminated under different light combinations (2b2r/2b2w/2r2w/4w) respectively harvested on day 7 and day 9 and the cell morphology of the cultures on day 9;

FIG. 5 illustrates the paramylon contents and biomass productivities of Euglena culture (day 7) cultivated with different ratios of CM medium to glucose as indicated as and the cell morphologies of Euglena cultures;

FIG. 6 illustrates the paramylon contents of Euglena cultures cultivated under medium combinations with enhancing or depleting different nitrogen sources;

FIG. 7 illustrates paramylon, protein contents and biomass productivities of Euglena cultures growing with medium supply under continuous 2b2r and 2b2w LED illumination which were harvested on day 9 and the cell morphologies of the Euglena cultures harvested on day 9 under microscope with 400× magnification;

FIG. 8(a) illustrates the Euglena dry biomass productivities and biomass composition analysis of Euglena culture cultivated under different LED conditions and fed-batch nutrient feeding as (Table 7) a 2-L bioreactor, FIG. 8 (b) shows the cell morphology (400×) of Euglena culture as FIG. 8 (a) on final day of harvest, FIG. 8 (c) shows the cell morphology (400×) of Euglena culture harvested on Days 0, 3 and 6 of 2L-T4 and T7 trials and FIG. 8 (d) shows the cell volume and dry weight estimation of Euglena cultures as FIG. 8 (a);

FIG. 9(a) illustrates the dry biomass productivity and paramylon content of 15L-scale Euglena cultivations under mixotrophic (MT) and heterotrophic (HT) conditions in a 20L bioreactor, FIG. 9 (b) illustrates the appearances and cell morphology (400×) of the bioreactor cultures as FIG. 9 (a), FIG. 9 (c) shows the dry biomass productivity and paramylon and protein contents of 15L-scale high protein Euglena cultivation and FIG. 9 (d) shows the cell morphology (400×) of high protein Euglena cultivation as (c) from days 2, 6, 9 and 12; and

FIG. 10 (a) illustrates the paramylon content of Euglena biomass of different pH-adjusted cultures, and FIG. 10 (b) and FIG. 10 (c) show paramylon contents of the cultures that were supplied with 0.5 vv/min air or 0.5 vv/min 40% oxygenated air, and 0.5 vv/min air or 2.5 vv/min air.

### DETAILED DESCRIPTION OF THE INVENTION

For the purposes of promoting and understanding the principles of the invention, reference will now be made to the embodiments illustrated in the drawings and described in the following written specification. It is understood that the present invention includes any alterations and modifications to the illustrated embodiments and includes further applications of the principles of the invention as would normally occur to one skilled in the art to which the invention pertains.

Accordingly, the teachings of the present invention, which relates to a method for producing axenicEuglena biomass with controlled biomass compositions, the method comprising the steps of: modifying components in an aqueous culture medium; culturing the Euglena using the modified aqueous medium under at least one culture condition in a fed-batch culture system; and harvesting the biomass when a desired amount of the controlled biomass compositions of interest is achieved.

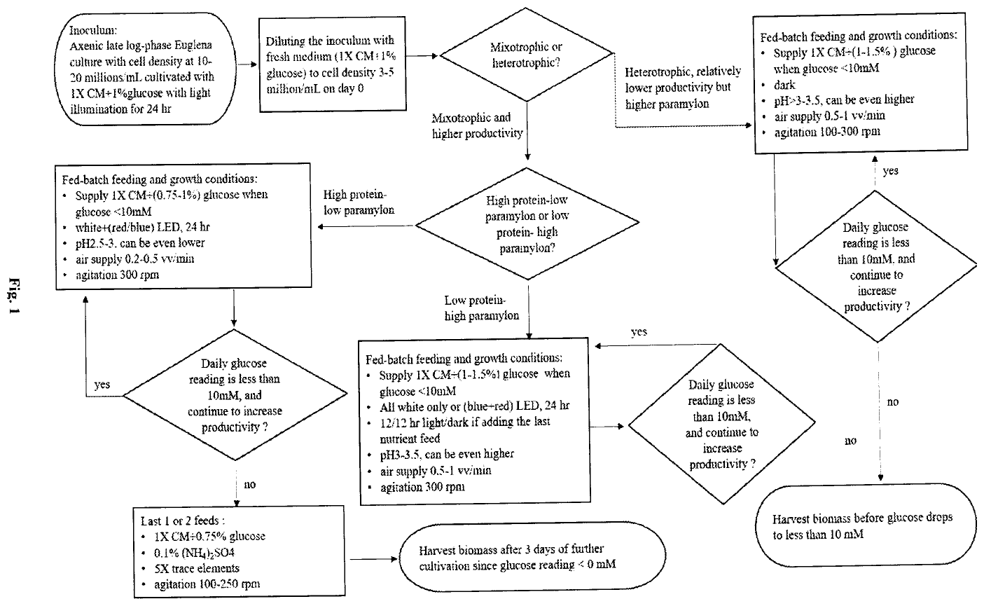

In one of the embodiments, the Euglena species is Euglena gracilis. A flow chart of the sequence of cultivation process to produce different Euglena gracilis biomass compositions with preferred feeding strategy is summarized in FIG. 1.

E. gracilis which generally has typical cell dimensions of 35-50 μm in length and 8-20 μm in diameter and naturally appears as elongated, spindle or sphere shapes which are closely related to biological clock, photosynthesis and environmental factors. In a natural complete life cycle, E. gracilis population of the same culture can display different morphologies dramatically from elongated shape under light-on hour, to spindle and nearly spherical shape at the end of light-off hour. When the cells are under different stressful conditions including exposures to UV, silver nano particles, aging, or less acidic condition (pH 5-8), exogenous plant hormones, they tend to be mostly appearing as spheric to gourd shapes. However, there is no further analysis on the cell volume in these different reports, though they are believed to be comparable in cell volume when comparing the micrographs of different shaped cells reported in these studies.

Based on FIG. 1, in order to obtain biomass compositions with higher paramylon content, the culture can be grown under heterotrophic culture condition with the following feeding strategy:

1×CM+(1 to 1.5%) glucose as a fed-batch medium, repeat feeding when glucose <10 mM, pH>3.0 or 3.5, air supply 0.5-1 vv/min and between agitation 100 to 300 rpm under dark condition. If daily glucose supply does not increase productivity anymore, the biomass can be harvested and if the productivity still increases, this feeding strategy is maintained with daily feeding of glucose until productivity stops rising, then the culture is ready for harvesting.

On the other hand, in order to obtain biomass compositions with higher amount of Euglena biomass, the culture can be grown under mixotrophic culture condition with the following feeding strategy:

1×CM+(1 to 1.5%) glucose as a fed-batch medium, repeat feeding when glucose <10 mM, light quality (all white/red and blue) for a period of 12 hours and 12 hours of darkness, pH>3.0 or 3.5; air supply 0.5-1 vv/min and between agitation 200 to 300 rpm. If daily glucose supply does not increase productivity anymore, the biomass can be harvested and if the productivity still increases, this feeding strategy is maintained with daily feeding of glucose until productivity stops rising, then the culture is ready for harvesting.

Alternatively, in order obtain biomass compositions with high protein content, the culture can be grown under mixotrophic culture condition with the following feeding strategy:

1×CM+(0.75 to 1%) glucose as a fed-batch medium, repeat feeding when glucose <10 mM, light quality (white and red or blue) for a period of 24 hours, pH 2.5-3.0 or lower; air supply 0.2-0.5 vv/min and between agitation 100 to 250 rpm. If daily glucose supply does not increase productivity linearly or significantly as of previous days, the culture medium is further enhanced with 1×CM+0.75% glucose, 0.1% (NH₄)₂SO₄ and 5× trace elements (TE) for the last 1-2 feed(s) and the biomass can be harvested within three days after the cultivation when the glucose level hits below 1 mM and if the productivity still increases, this feeding strategy is maintained with daily feeding of CM medium and glucose until productivity stops rising, then the culture can be further enhanced as above mentioned and is ready for harvesting.

In one of the embodiments, the method further comprises a feedback loop mechanism. The feedback loop mechanism comprises determining whether the biomass contains the desired amount of biomass compositions of interest before harvesting the biomass; and repeating modifications on the aqueous culture medium to increase production of biomass compositions of interest if the biomass does not contain the desired amount of compositions.

In a preferred embodiment of the present invention wherein the desired amount of the controlled biomass compositions is at least 65 g/L with cell density of at least 40 millions/mL.

In another preferred embodiment, the repeating of modifications on the aqueous culture medium further comprises: adding, depleting, and enhancing nutritional components of the culture medium, wherein, the adding of nutritional components includes feeding additional nutrients to the modified culture medium is required when the glucose level drops between 0-10 mM for maintaining or elevating productivity of the biomass, and its compositions, and the enhancing of nutritional components includes higher concentration of trace elements and nitrogen sources.

In a preferred embodiment, the adding of nutritional components further includes but not limited to a higher culture medium to glucose ratio, wherein, the ratio is 1× culture medium to 0.75%-1.5% glucose.

In another preferred embodiment, the enhancing of nutritional components further includes but not limited increasing the trace elements by five times with 0.1% ammonium sulphate for producing protein-rich biomass.

In a preferred embodiment, the biomass compositions constitute of lipids, protein content with at least 50% of dry biomass; or paramylon composition with at least 60% of dry biomass.

In another preferred embodiment, in analyzing the biomass compositions, paramylon composition is utilized as an inverse indicator to predict protein content and cell morphology or cell volume is used to predict protein and paramylon compositions in the biomass. For example, protein content can be predicted with the inverse indicator wherein low paramylon content indicates high protein content and vice versa.

In another preferred embodiment, the predicting of protein and paramylon compositions based on cell morphology comprises: monitoring the volume and shape of the cells; wherein small and elongated cells indicate lower paramylon content with higher protein content, and large and spherical cells indicate higher paramylon content with lower protein content.

The above embodiments provide an additional advantage to the present invention wherein the method does not give apparent reduction of biomass productivity even keeping cell incubation for several days after stationary phase. With several trials of high productivity cultivations, the interrelationship of macronutritional compositions of Euglena is established. It is proposed that an analyzed paramylon content (%) of a Euglena biomass sample can be used as an indicator to predict the protein contents at a reasonable range. For example, low paramylon content indicates high protein content in the biomass and vice versa.

The present invention also allows the analysis of an average Euglena cell volume from wet biomass and nutritional compositional analysis of biomass from different cultivation trials under different conditions, and it was found that there is a positive correlation between cell volume and paramylon content amount wherein the larger the cell size is more attributed by paramylon content than protein or lipid contents.

In one of the embodiments, the culture condition comprises photoautotrophic, heterotrophic, mixotrophic or any combination thereof.

In a preferred embodiment, the culturing of the Euglena further comprises: adjusting the lighting setting and spectrum based on the culture conditions that require the presence of light wherein, said light setting and spectrum is a combination of white and red or white and blue for obtaining higher protein compositions and all white or a combination of blue and red for obtaining higher paramylon compositions. In the presence of light, the light quality is adjusted to white LED light and in combination with red or blue LED light wherein the microalgae cultivation is exposed to 24 hours of light. Whereas under the mixotrophic culture condition, the Euglena cultivation is exposed to 12 hours of light and 12 hours of darkness. In the absence of light or under heterotrophic culture condition, the Euglena cultivation is kept in darkness for a period of 24 hours. Due to the different light conditions, the productivity of the E. gracilis will vary and it will greatly affect the types of compositions produced.

In view of the above, cultivations under mixotrophical and heterotrophical are anticipated and theoretically possible to have theoretically the highest productivity of Euglena culture of more than 300 g/L. In another preferred embodiment, the feeding of additional nutrients to the modified culture medium is required when the glucose level drops between 0-10 mM to maintain or elevate productivity of the biomass and its compositions. The feeding strategy will greatly affect the biomass composition production.

The present invention also relates to a system for producing Euglena biomass with controlled biomass compositions, the system comprises: Euglena culture grown in a modified aqueous culture medium under at least one culture condition; wherein the system is in a feedback loop mechanism; and at least one nutrient feeding strategy fed to the culture grown in the modified aqueous culture medium during feedback loop mechanism for desired biomass compositions.

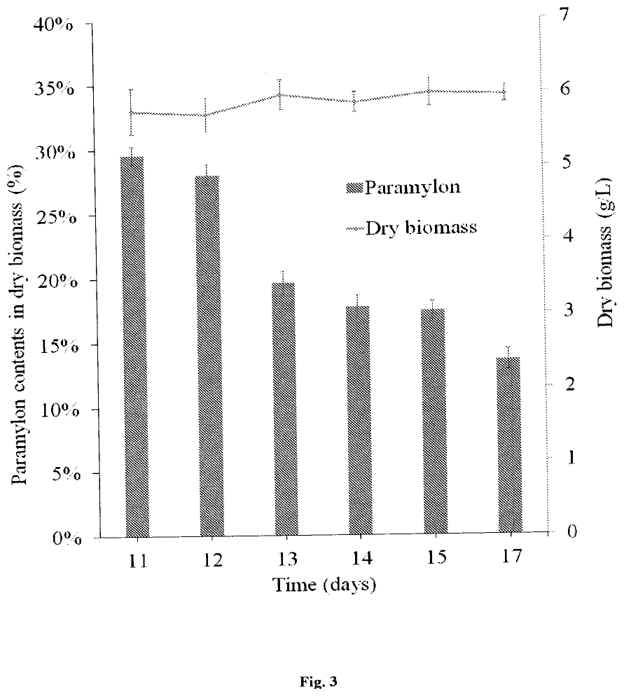

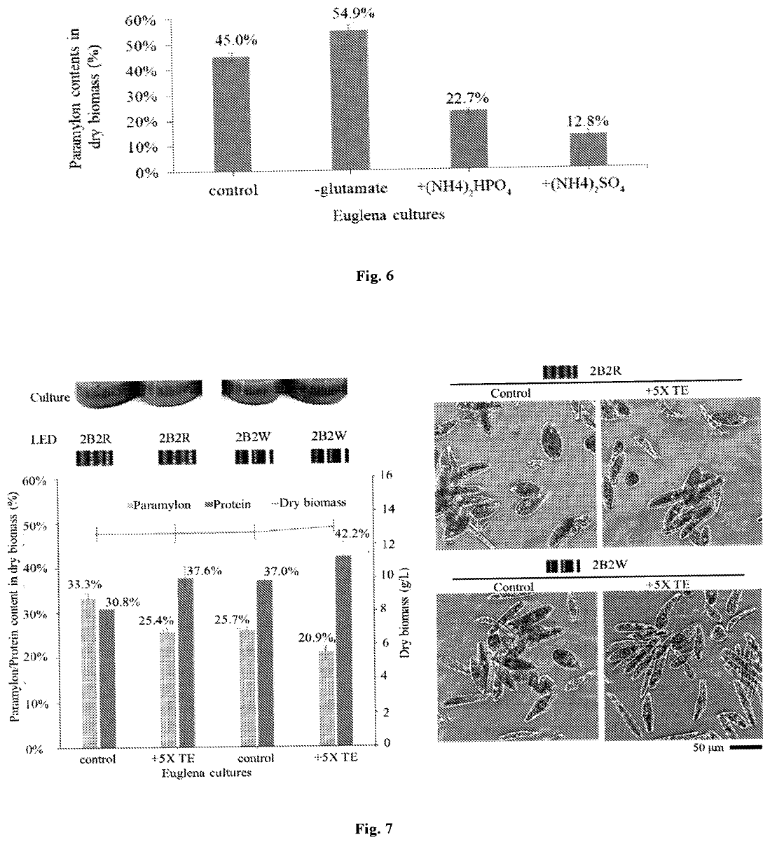

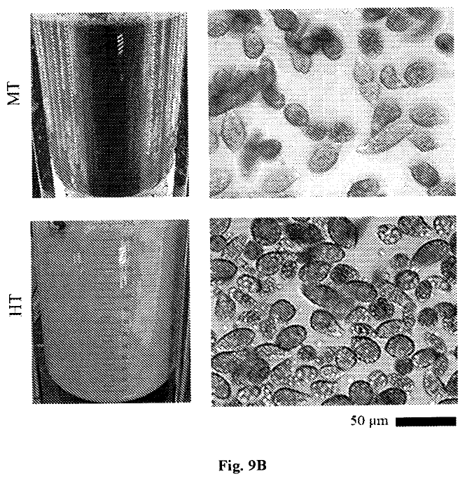

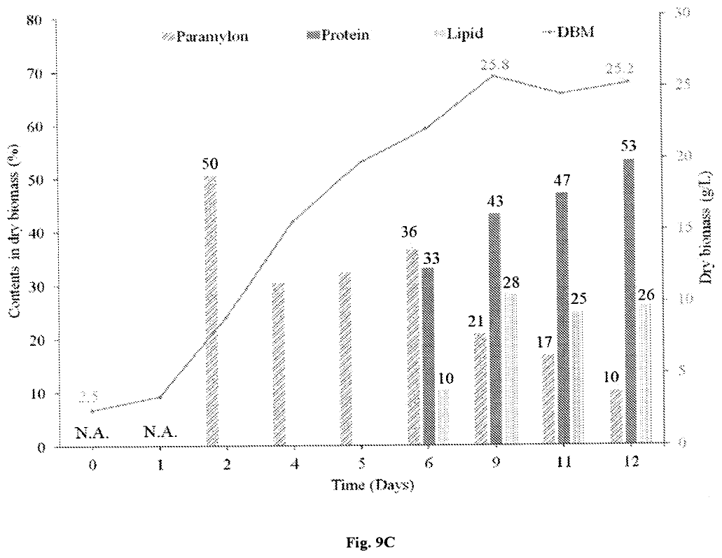

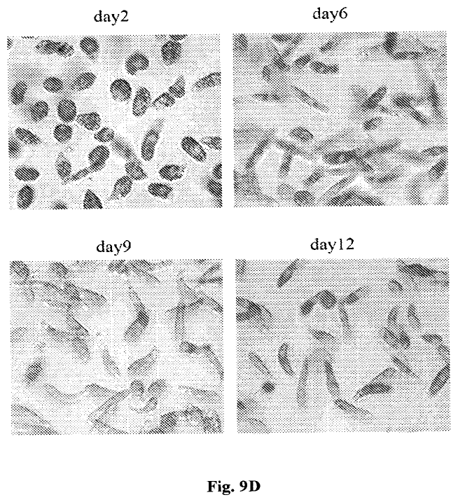

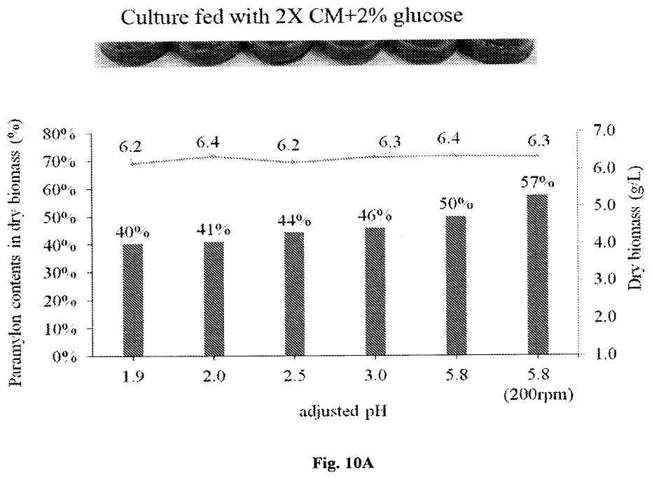

In another embodiment of the present invention, the nutrient feeding strategy includes adding, depleting, and enhancing nutritional components of the culture medium. Depleting different components of medium such as glucose or any of the medium's component could produce different results. For example, by depleting glutamate as a nitrogen source in the medium as shown in FIG. 6 (-glutamate), the paramylon content in dry biomass will be higher, but productivity is still comparable to other samples in the same test. On the other hand, protein content will be lower, as paramylon has occupied the higher content (%) of the dry biomass. If glucose is depleted earlier than the depletion of culture medium during cultivation, this would cause the dry biomass to have a higher protein content after further incubation. For example, in FIG. 9c, the glucose content on days 7-9 should have been consumed/depleted, while the medium/Nitrogen source components were still available, and as the culture after day 9 continued to grow up to day 11, the protein content became higher, but the paramylon content became lower in the biomass. However, in this period, the biomass productivity remained relatively unchanged at ˜25 g/L. FIG. 3 is another similar experiment, wherein glucose is depleted (used up) by the cell culture since day 11.

In one of the embodiments, the light setting and spectrum are adjusted based on the culture condition, and the culture condition comprises photoautotrophic, heterotrophic, mixotrophic or any combination thereof.

In view of the above, the present invention provides another advantage wherein the biomass compositions can be tuned or controlled, making it a more flexible method and system for scaling up production, at the same time increasing productivity of the Euglena biomass efficiently.

An example for the analysis of average Euglena cell volume from wet biomass and nutritional compositional analysis of biomass

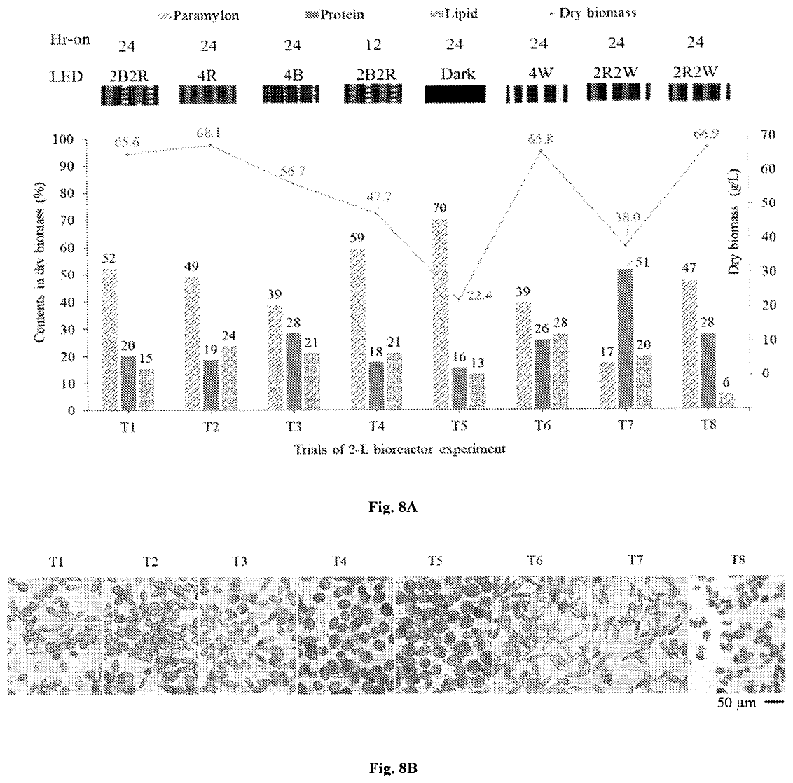

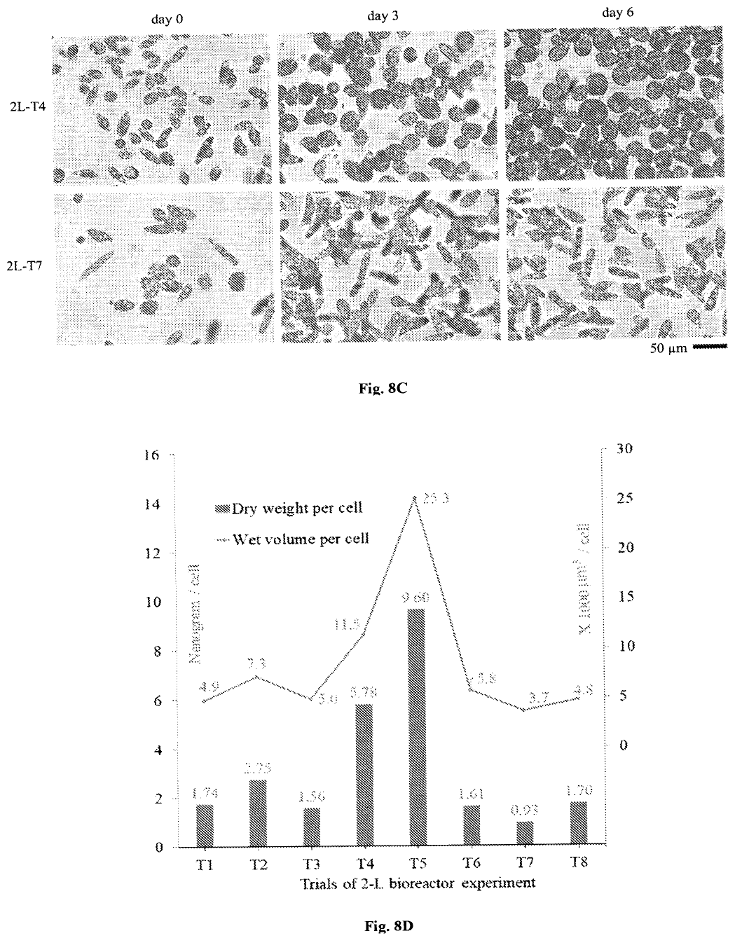

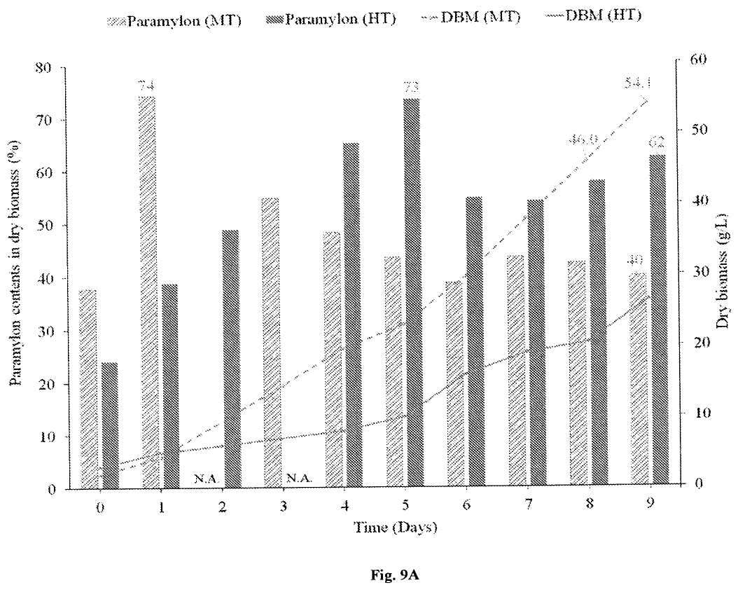

Euglena cell volumes under different cell culture media were reported to be around 2980 to 3410 μm³ which is a cell volume range comparable to the 2L-T7 cell size estimated to be 3700 μm³ based on fresh wet biomass volume. This can be seen in FIG. 8d and Table 1 below.

TABLE 1

| | Content per cell | | | | | | | | | | | |
|---|---|---|---|---|---|---|---|---|---|---|---|---|
| | | | | | Wet | | | | | | | |
| | | | | Dry | cell | | Relative content per cell compared to T7's (assigned as 1s) | | | | | |
| | Paramylon | Protein | Lipid | cell | volume | | | | | | | Wet |
| | content | content | content | weight | (X1000 | OD₄₈₀/ | Paramylon | Protein | Lipid | OD₄₈₀/ | Dry cell | cell |
| Trial | (ng/cell) | (ng/cell) | (ng/cell) | (ng) | μm³) | OD₇₅₀ | content | content | content | OD₇₅₀ | weight | volume |
| 2L-T1 | 0.91 | 0.35 | 0.27 | 1.74 | 4.9 | 1.16 | 5.74 | 0.74 | 1.47 | 0.78 | 1.88 | 1.34 |
| 2L-T2 | 1.35 | 0.51 | 0.65 | 2.75 | 7.3 | 1.13 | 8.52 | 1.08 | 3.57 | 0.76 | 2.96 | 2.01 |
| 2L-T3 | 0.61 | 0.44 | 0.33 | 1.56 | 5.0 | 1.18 | 3.83 | 0.94 | 1.82 | 0.79 | 1.69 | 1.38 |
| 2L-T4 | 3.43 | 1.02 | 1.22 | 5.78 | 11.5 | 1.09 | 21.64 | 2.15 | 6.73 | 0.74 | 6.24 | 3.15 |
| 2L-T5 | 6.72 | 1.49 | 1.28 | 9.60 | 25.3 | 1.02 | 42.40 | 3.15 | 7.05 | 0.69 | 10.36 | 6.92 |
| 2L-T6 | 0.63 | 0.41 | 0.45 | 1.61 | 5.8 | 1.17 | 3.99 | 0.88 | 2.46 | 0.79 | 1.74 | 1.60 |
| 2L-T7 | 0.16 | 0.47 | 0.18 | 0.93 | 3.7 | 1.49 | 1.00 | 1.00 | 1.00 | 1.00 | 1.00 | 1.00 |
| 2L-T8 | 0.81 | 0.47 | 0.10 | 1.70 | 4.8 | 1.28 | 5.09 | 1.00 | 0.53 | 0.86 | 1.84 | 1.31 |

2L-T7 is the only cultivation trial to produce highest protein, low paramylon and high cell density based on the present invention under preferred conditions. However, other trials are found to have larger cell volumes than previous report, and these volumes can range from 3900 μm³ to 9600 μm³. From the nutritional composition analysis of 2L-T1 to T8 as seen in FIG. 8a, Table 1, it was found that there is a correlation between increasing paramylon content per cell (0.16 to 6.72 ng/cell) with cell volume increase per cell (from 3700 to 25000 μm³/cell), meanwhile protein content per cell remains relative stable (0.45 to 1.49 ng).

It was also found that cell morphology and cell volume have a correlated relationship with each other as seen in FIGS. 8c, 8d and Table 1 with small cells, as T7 (see FIGS. 8c, 8d) tend to be more elongated with higher chlorophyll absorbance (OD₄₈₀/OD₇₅₀) and bigger cells tend to be more spherical (T4 and T5) with lower chlorophyll absorbance.

An example of the materials and method used are further illustrated below:

The medium used for this invention was modified and simplified based on Cramer and Myers Medium (CM) of a previous study, as it was found that some of the components are non-essential for increasing Euglena productivity in the cultivation trials. The standard 1× modified CM medium in this invention includes the following components: 1 g/L (NH₄)₂HPO₄, 1 g/L KH₂PO₄, 0.2 g/L MgSO₄·7H₂O, 0.02 g/L CaCl₂; and trace elements as follows, 3 mg/L Fe₂(SO₄)₃·nH₂O, 2.48 mg/L H₃BO₃, 1.8 mg/L MnCl₂·4H₂O, 1.5 mg/L CoSO₄·7H₂O, 0.4 mg/L ZnSO₄·7H₂O, 0.2 mg/L Na₂MoO₄·2H₂O, 0.02 mg/L CuSO₄·5H₂O; and vitamins as follows, 0.5 mg/L Vitamin B1, 0.02 mg/L Vitamin B₁₂.

The sodium citrate (0.8 g/L) in the original CM medium was replaced with 0.75 g/L citric acid (C₆H₈O₇·H₂O) to provide a lower preferrable pH, thus the least amount of additional acid is required when preparing concentrated modified CM medium (pH 3). The modified CM medium can be prepared as 25-50 (25-50×) times concentrated depending on the needs of the experiment. The pH of modified CM medium is finally adjusted with mineral acid to pH 3-3.5 before autoclave at 121° C. for 20 minutes. The amount of glutamic acid in the modified CM medium can be included from 0 to 1 g/L depending on the requirement of the designed experiment. It is included into the modified CM for autoclaving when less concentrated CM medium (25×-35×) is to be prepared. For mixotrophic or heterotrophic cultivations, glucose as another main component for culture medium was prepared separately as 50%-80% weight-to-volume solution. The modified CM and glucose were diluted to be 1×CM and 1% glucose for Euglena culture maintenance as well as for fed-batch cultivation of both mixotrophic and heterotrophic mode. When performing fed-batch cultivation, the glucose content in a culture is confirmed with blood glucose test kit. If the glucose concentration in the culture is close to 5 mM or even 0 mM, new concentrated CM and glucose (1× to 1%, depends on preferred Euglena nutritional composition) are added into culture for further growth.

Mature Euglena culture was inoculated into fresh medium (1×CM+1% glucose) at the ratio of 1/10 volume of fresh medium in a 250 mL/1 L Erlenmeyer flask. The fresh culture generally has cell density of 1 million/mL and is kept 15 cm away from the LED panel, which will be illuminated with photon intensity of 100-200 μmol m⁻²s⁻¹). The quality of light spectrum (all white, half-blue-half-red, half-blue/red-half-white) was to be varied depending on what kind of Euglena biomass composition is preferred to produce. Generally, the new culture will regrow to 7-10 millions/mL.

To maintain inoculum, the Euglena culture in an Erlenmeyer flask (250 mL) did not require additional air supply if the volume of culture occupies 50% of total flask volume. When performing cultivation in 2 L or 20 L bioreactor, the aeration rate was around 0.5-1 vv/min.

The pH of culture can be maintained at a range from pH 2.0 to 3.5. The adjustment of pH is not necessary once the culture was inoculated with CM medium (pH 3.5) and cultivations kept ongoing, as Euglena culture release acidic metabolite to lower the pH of the medium to be less than 3.

The temperature of cultivations was mostly kept at 28° C. with chiller.

To maintain inoculum, the Euglena culture in an Erlenmeyer flask (250 mL) did not require agitation if the volume of culture occupied 50% of total flask volume. When performing cultivation in 2 L or 20 L bioreactor, the agitation rate was kept at 100-300 rpm.

Another example of production of Euglena biomass of different nutritional composition without losing biomass productivity after stationary phase is described below:

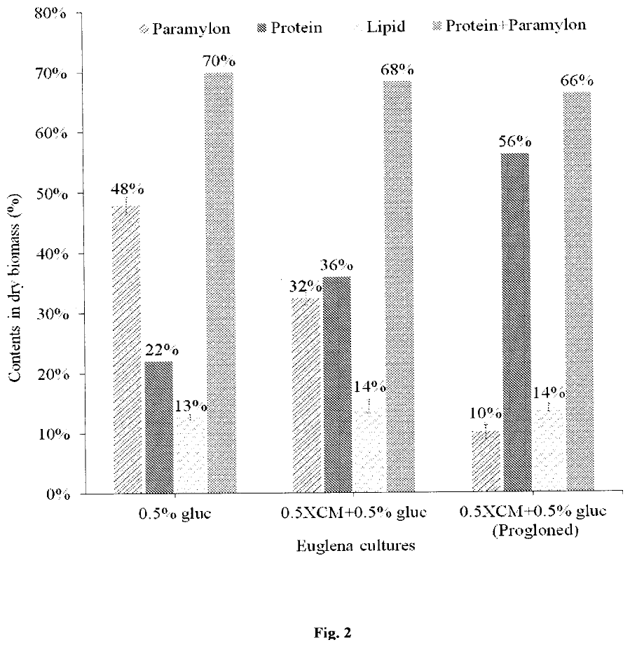

Referring to FIG. 2 and Table 2 below, paramylon or protein ratio in Euglena biomass can be tuned depending on medium and cultivation duration, though it took as long as 20 days to cultivate. The protein can be as high as 55% at ˜6g/L and the daily yield or productivity remained stable from day 11 to 20 as shown in FIG. 3.

TABLE 2

| | Nutrient feeding formula | | Day of |
|---|---|---|---|
| Euglena culture | Day 0 | Days 6 and 8 | Harvest |
| Higher [Glucose] | 1XCM + 0.5% | 0.5% glucose | day 11 |
| | glucose | | |
| Control | 1XCM + 0.5% | 0.5XCM + 0.5% | day 11 |
| | glucose | glucose | |
| Prolonged | 1XCM + 0.5% | 0.5XCM + 0.5% | day 20 |
| | glucose | glucose | |

It was established that the inversely proportional relationship of protein and paramylon content exists in different samples of Euglena, and this allows us to predict protein content based on paramylon content of an Euglena sample for further study to develop a method of producing high protein content Euglena biomass with shorter period of cultivation and higher productivity.

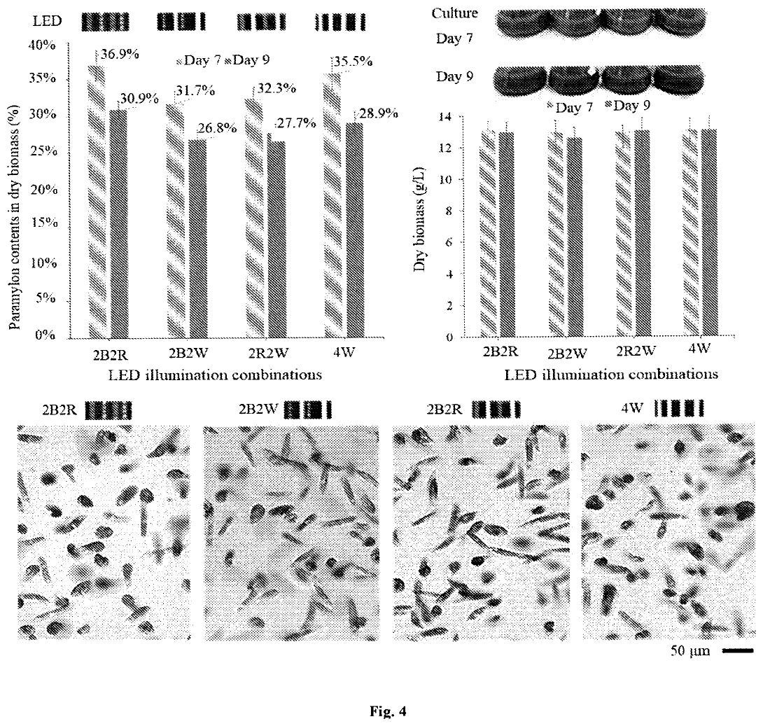

Previously, it was reported that the paramylon content and dry biomass of E. gracilis peaked at the 48^{th} hour after inoculation and both dropped continuously after the time point (up to the 144^{th} hour). By referring to FIGS. 3 and 4, the present invention shows that Euglena biomass would not drop even the cell passed log phase and stayed at stationary phase for a long period (>7 days).

An example of producing Euglena biomass with higher protein within a shorter period of time and a higher biomass productivity with more preferred cultivation conditions is illustrated below:

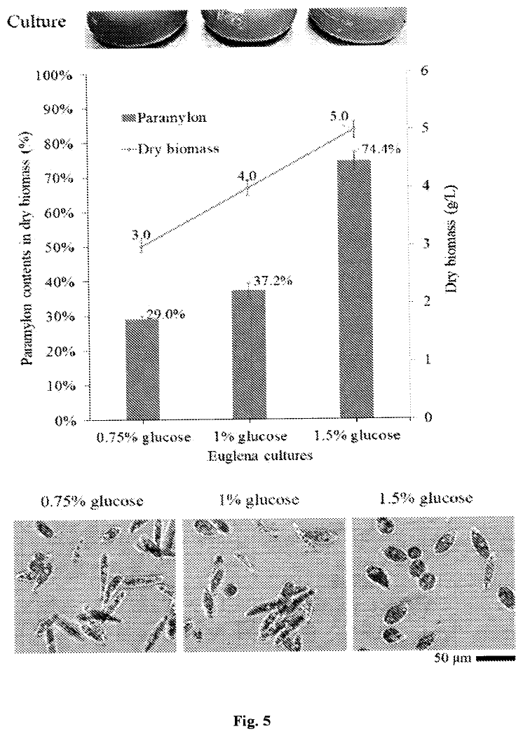

Firstly, a fed-batch cultivated Euglena culture with higher CM to glucose ratio such as 1×CM to 0.75% glucose as seen in Table 3 and FIG. 5, FIG. 8a Trial T7 is more preferred.

TABLE 3

| | Nutrient feeding | Day of |
|---|---|---|
| Euglena culture | formula on day 0 | Harvest |
| 0.75% glucose | 1XCM + 0.75% glucose | day 7 |
| 1% glucose | 1XCM + 1% glucose | day 7 |
| 1.5% glucose | 1XCM + 1.5% glucose | day 7 |

Secondly, additional nitrogen sources in the CM medium for final nutrient feed as seen in Table 4, FIG. 6 and FIG. 8a Trial T7 are more preferred.

TABLE 4

| Euglena | Nutrient feeding | Day of |
|---|---|---|
| culture | formula on day 0 | Harvest |
| control | 2XCM + 2% glucose | day 7 |
| −glutamate | 2XCM − glutamate + 2% glucose | day 7 |
| +(NH4)2HPO4 | 2XCM + 2% glucose + | day 7 |
| | 0.2% (NH₄)₂HPO₄ | |
| +(NH4)2SO4 | 2XCM + 2% glucose + | day 7 |
| | 0.1% (NH₄)₂HPO₄ + | |
| | 0.1% (NH₄)₂SO₄ | |

Thirdly, additional trace elements in the CM medium for final nutrient feed as seen in Table 5, FIG. 7, FIG. 8a Trial T7 are more preferred.

TABLE 5

| Euglena | LED | Nutrient feeding | Day of |
|---|---|---|---|
| culture | condition | formula on days 0 and 4 | Harvest |
| control | 2b2r | 2XCM + 2% Gluc | day 9 |
| +5X TE | 2b2r | 2XCM + 2% Gluc + 5X TE | day 9 |
| control | 2b2w | 2XCM + 2% Gluc | day 9 |
| +5X TE | 2b2w | 2XCM + 2% Gluc + 5X TE | day 9 |

Fourth, a mixotrophic cultivation is necessary and 2b2w or 2r2w LEDs are preferred for 24-hour illumination as shown in Table 6 and FIG. 4.

TABLE 6

| LED | Nutrient feeding formula | Day of |
|---|---|---|
| illumination | Day 0 and 4 | Harvest |
| 2b2r | 2XCM + 2% glucose | day 7 and 9 |
| 2b2w | 2XCM + 2% glucose | day 7 and 9 |
| 2r2w | 2XCM + 2% glucose | day 7 and 9 |
| 4w | 2XCM + 2% glucose | day 7 and 9 |

Incubated additional 2 days or longer of cultivations after glucose in medium is consumed before harvest and this is tabulated in Table 6 and FIG. 9c.

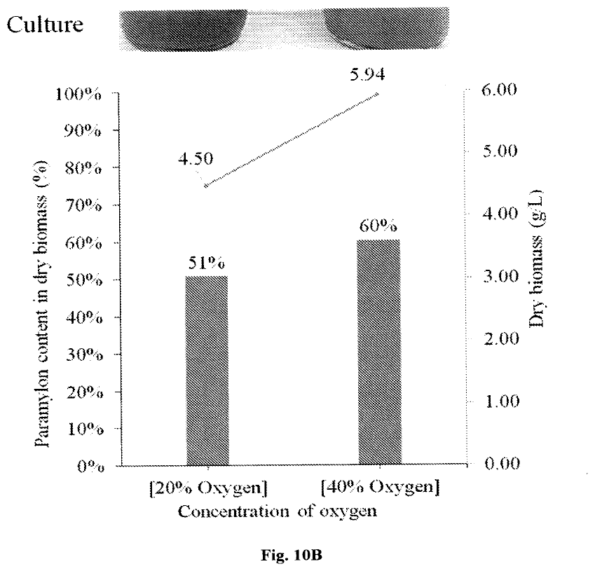

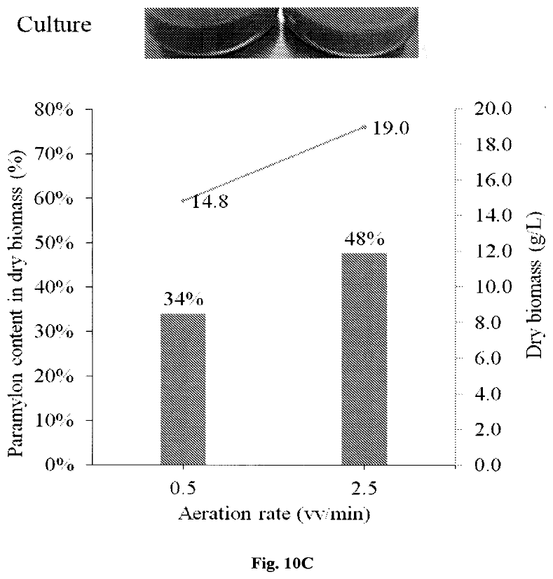

It was found in previous findings that pH close to 7 can facilitate higher protein content in Euglena, it was found in the present invention that lower pH is more preferred for lower paramylon contents and thus implying higher protein contents. The same is illustrated in FIG. 10a. The pH adjustment of the culture is not required if pH of culture is in the range of 2 to 3 as Euglena culture release acidic metabolite to lower the pH of the medium to be less than 3.

A lower to mild aeration rate for lower DO₂ environment for Euglena culture growth is preferred, as seen in FIG. 10c.

An example of producing Euglena biomass with higher paramylon content:

Firstly, mixotrophic cultivation is unnecessary. However, if mixotrophic mode is used for higher productivity and higher paramylon content, then all white or 2b2r LEDs are preferred for 24-hour illumination as shown in Table 7 and FIG. 4 or 12 hr-on and 12 hr-off and as seen in FIG. 8a Trial T4. In view of the above, heterotrophic cultivation is more preferred and this is depicted in FIG. 8a Trial T5.

TABLE 7

| | LED | Nutrient | | | Residual |
|---|---|---|---|---|---|
| | property/ | formula for | | Cell | glucose |
| | light-on | fed-batch | Day of | density | (mM) on day |
| Trial | Hr | cultivation | Harvest | (mill/mL) | of harvest |
| 2L-T1 | 2B2R/24 | 1X CM + 1% | day 6 | 37.7 | 0 |
| | | glucose | | | |
| 2L-T2 | 4R/24 | 1X CM + 1% | day 6 | 24.8 | 16 |
| | | glucose | | | |
| 2L-T3 | 4B/24 | 1X CM + 1% | day 6 | 36.3 | 0 |
| | | glucose | | | |
| 2L-T4 | 2B2R/12 | 1X CM + 1% | day 7 | 8.3 | 48 |
| | | glucose | | | |
| 2L-T5 | Dark/24 | 1X CM + 1% | day 10 | 2.3 | 67 |
| | | glucose | | | |
| 2L-T6 | 4W/24 | 1X CM + 1% | day 6 | 40.9 | 0 |
| | | glucose | | | |
| 2L-T7 | 2R2W/24 | 1X CM + | day 9 | 41 | 0 |
| | | 0.75% glucose^{a} | | | |
| 2L-T8 | 2R2W/24 | 1X CM + 1% | day 7 | 39.3 | 0 |
| | | glucose | | | |

Note:
^{a}On day 5, the last dose of nutrient feed contained extra 5X trace elements, 0.1% (NH₄)₂HPO₄ and 0.1% (NH₄)₂SO₄.

Then Euglena culture is harvested before glucose in the culture medium is fully consumed and this is tabulated in Table 7, FIG. 4, FIGS. 8a-b Trials 2,4,5.

Then, fed-batch cultivated Euglena culture with lower CM to glucose ratio such as 1×CM to 1.5% glucose as tabulated in Table 3 and depicted in FIG. 5.

At the end of cultivation, higher pH is preferred for higher paramylon content (see FIG. 10a).

A higher oxygen concentration during the cultivation of Euglena is more preferred for higher paramylon content (see FIG. 10b).

A higher aeration rate for higher DO₂ environment for Euglena culture growth is preferred (see FIG. 10c).

Besides increasing the cell culture scale as seen in FIGS. 9a-d for increasing the productivity with abovementioned method in FIG. 1, and by combining mixotrophic and heterotrophic cultivations with the feeding strategies, it is expected that theoretically the highest productivity (>300 g/L) can be achieved as the same could be predicted based on the cell volume/dry weight (as seen in FIGS. 8b-d and Table 1) of mixotrophic culture to heterotrophic culture, the heterotrophic cell volume and cell dry weight (Table 1 and Trial T5) can have at least 3 to 10 times more than that of mixotrophic culture.

Scalability of mixotrophic and heterotrophic production (20 L)

The scalability of 2 L trials to a larger scale by more than 10 times of culture volume was further tested by fed-batch cultivations using the same nutrient formula as 2 L bioreactor tests (Table 1). Three trials of around 15-liter working volume cultures in 20 L bioreactor, respectively under mixotrophic (MT, 1×CM+1% glucose), heterotrophic (HT, 1×CM+1% glucose) and high-protein modes (HP, 1×CM+0.75% glucose), were performed and analyzed. Under MT/HT and HP cultivation modes, their dry biomass productivities reached ˜54, 26 and 25 g/L. Also, their paramylon contents in dry basis peaked on day 2 to 4(early log phase). The cell morphologies of these trials were observed to be mostly oval shape (FIG. 9b) for MT and HT culture due to their higher paramylon contents (42-61% on day 9). For HP cultivation, the cell morphology transformed from mostly oval shape on day 0 to be progressively more slender and elongated shape on days 6-12 with the gradual decrease in paramylon (low to 10%) but increase in protein contents (up to 53%) in DBM.

The present invention explained above is not limited to the aforementioned embodiment and drawings, and it will be obvious to those having an ordinary skill in the art of the prevent invention that various replacements, deformations, and changes may be made without departing from the scope of the invention.

1. A method for producing axenic Euglena biomass with controlled biomass compositions, the method comprising the steps of:
modifying components in an aqueous culture medium;
culturing the Euglena using the modified aqueous medium under at least one culture condition in a fed-batch culture system; and,
harvesting the biomass when a desired amount of the controlled biomass compositions of interest is achieved.

2. The method of claim 1 wherein, the Euglena species is Euglena gracilis.

3. The method of claim 1, wherein the method further comprises a feedback loop mechanism.

4. The method as claimed in claim 3, wherein the feedback loop mechanism comprises:
determining whether the biomass contains the desired amount of biomass compositions of interest before harvesting the biomass; and
repeating modifications on the aqueous culture medium to increase production of biomass compositions of interest if the biomass does not contain the desired amount of compositions.

5. The method of claim 1, wherein the desired amount of the controlled biomass compositions is at least 65 g/L with cell density of at least 40 millions/mL.

6. The method of claim 4, wherein the repeating of modifications on the aqueous culture medium further comprises:
adding, depleting, and enhancing nutritional components of the culture medium, wherein,
the adding of nutritional components includes feeding additional nutrients to the modified culture medium is required when the glucose level drops between 0-10 mM for maintaining or elevating productivity of the biomass and its compositions, and,
the enhancing of nutritional components includes higher concentration of trace elements and nitrogen sources.

7. The method of claim 6, wherein the adding of nutritional components further includes but not limited to a higher culture medium to glucose ratio, wherein, the ratio is 1× culture medium to 0.75%-1.5% glucose.

8. The method of claim 6, wherein the enhancing of nutritional components further includes but not limited increasing the trace elements by five times with 0.1% ammonium sulphate for producing protein-rich biomass.

9. The method of claim 1, wherein the biomass compositions constitute of lipids, protein content with at least 50% of dry biomass; or paramylon composition with at least 60% of dry biomass.

10. The method of claim 9, wherein the paramylon composition is utilized as an inverse indicator to predict protein content and cell morphology or cell volume is used to predict protein and paramylon compositions in the biomass.

11. The method of claim 10, wherein the predicting of protein and paramylon compositions based on cell morphology comprises:
monitoring the volume and shape of the cells;
wherein small and elongated cells indicate lower paramylon content with higher protein content, and large and spherical cells indicate higher paramylon content with lower protein content.

12. The method of claim 1, wherein the culture condition comprises photoautotrophic, heterotrophic, mixotrophic or any combination thereof.

13. The method of claim 1, wherein the culturing of the Euglena further comprises:
adjusting the lighting setting and spectrum based on the culture conditions that require the presence of light wherein,
said light setting and spectrum is a combination of white and red or white and blue for obtaining higher protein compositions and all white or a combination of blue and red for obtaining higher paramylon compositions.

14. A system for producing Euglena biomass with controlled biomass compositions, the system comprising:
Euglena culture grown in a modified aqueous culture medium under at least one culture condition;
wherein the system is in a feedback loop mechanism; and,
at least one nutrient feeding strategy fed to the culture grown in the modified aqueous culture medium during feedback loop mechanism for desired biomass compositions.

15. The system of claim 14, wherein the nutrient feeding strategy includes adding, depleting, and enhancing nutritional components of the culture medium.

16. The system of claim 14, wherein the light setting and spectrum are adjusted based on the culture condition, and the culture condition comprises photoautotrophic, heterotrophic, mixotrophic or any combination thereof.
